(19) United States
(12) Patent Application Publication
(10) Pub. No.: US 2022/0176165 A1
(43) Pub. Date: Jun. 9, 2022

(54) THERAPEUTIC SOUND AND DIRECTED SOUND TRANSMISSION SYSTEMS AND METHODS

(71) Applicant: Zaps Labs Inc., Austin, TX (US)

(72) Inventors: Joseph Frank Scalisi, Lakeway, TX (US); Adrian Simon Lanch, Lakeway, TX (US)

(21) Appl. No.: 17/535,115

(22) Filed: Nov. 24, 2021

Related U.S. Application Data
(63) Continuation-in-part of application No. 17/364,716, filed on Jun. 30, 2021, now Pat. No. 11,256,878.
Application No. 17/364,716 is a continuation-in-part of application No. 17/482,764, filed on Sep. 23, 2021.
Application No. 17/482,764 is a continuation-in-part of application No. 17/506,469, filed on Oct. 20, 2021.
(60) Provisional application No. 63/121,851, filed on Dec. 4, 2020.

(51) Int. Cl. A61N 7/00; H04R 1/22

(52) U.S. Cl. CPC A61N 7/00; A61N 2007/0004; A61N 2007/0078; H04R 1/227

(57) ABSTRACT

A method is provided for delivering directed transmission of sound waves through modulation on an ultrasonic carrier. In some embodiments, the method comprises connecting at least one directed sound source to an audio system and emitting, via the at least one directed sound source, audio from the audio system, wherein the emitting comprises emitting medium frequency audio sound waves and higher frequency audio sound waves. The audio may be selected via a master control unit, operatively coupled to a mobile application. In some embodiments, a first audio selection is configured to be heard only through a first directed sound source, and a second audio selection is configured to be heard only through a second directed sound source. Additionally, systems and methods are provided for delivering therapeutic sound, through a sinusoidal signal, within an environment intended to cause resonance in certain cells.

### CROSS-REFERENCE TO RELATED APPLICATIONS

The entire contents of the following application are incorporated by reference herein: U.S. Provisional Patent Application No. 63/121,851; filed Dec. 4, 2020; and entitled DIRECTED SOUND TRANSMISSION SYSTEMS AND METHODS.

The entire contents of the following application are incorporated by reference herein: U.S. Provisional Patent Application No. 17/364,716; filed Jun. 30, 2021; and entitled DIRECTED SOUND TRANSMISSION SYSTEMS AND METHODS.

The entire contents of the following application are incorporated by reference herein: U.S. Provisional Patent Application No. 17/482,764; filed Sep. 23, 2021; and entitled DIRECTED SOUND TRANSMISSION SYSTEMS AND METHODS USING POSITION LOCATION.

The entire contents of the following application are incorporated by reference herein: U.S. Provisional Patent Application No. 17/506,469; filed Oct. 20, 2021; and entitled DIRECTED SOUND TRANSMISSION SYSTEMS AND METHODS.

### BACKGROUND

### Field

Various embodiments disclosed herein relate to speakers. Certain embodiments relate to parametric speakers.

Time spent sitting or resting can be most effectively used while multi-tasking or receiving treatment of some kind. Non-invasive therapy, which requires little set-up time, represents the best use of time activity while sitting or resting.

In a COVID-19 world, therapies that can be delivered contactless are worth their weight in gold. Such may provide a present and further benefit during what may be described as a passive activity. It is well-known that matter is responsive to resonant energy on a molecular and atomic scale. Resonant energy can cause cell walls to explode and covalent bonds to break. For instance, energy applied at a resonant frequency, selective to and corresponding to certain cancers, has resulted in the destruction of those cancer cells while healthy cells are left mainly unaffected. Achieving a resonant frequency for matter, such as particular types of cells, depends on factors that include the transmissibility of the particular matter to a given input driving frequency. Transmissibility, which may be determined by EQ. 1, characterizes the mechanical response of a particular matter to a given input driving frequency.

Transmissibility=1/square root [(1−ω²/ωₙ²))²+(2ξω/ωₙ)²] (EQ. 1)

As shown by EQ.1, transmissibility is a function of the angular input driving frequency ω of the energy delivered to a particular matter, the angular resonant frequency ωₙ of that matter, and the damping coefficient ξ of that matter. Damping describes the absorption of energy oscillations. Different resonant frequencies and a corresponding damping coefficient may exist for a given matter, material, cell type, etc. Transmissibility increases with a decreasing damping coefficient value. Consequently, matter having the lowest damping coefficient may receive the greatest resonant response to a resonant driving frequency.

In view of the above, a need exists to better facilitate the conveyance of sound in various environments while delivering contactless therapies that may improve health.

### SUMMARY

The disclosure includes a method for providing sound and therapeutic treatment in a listening environment. In some embodiments, the method comprises modulating one or more ultrasonic pressure waves by audio content to produce one or more modulated carrier signals and sending the one or more modulated carrier signals, to one or more target locations in the listening environment, through a transmission medium, wherein in connection with the one or more ultrasonic pressure waves reaching the one or more target locations, the one or more modulated carrier signals demodulate. In some embodiments, the method includes directing a sinusoidal signal at the one or more target locations in the listening environment, wherein a frequency of the sinusoidal signal produces resonance in a malignant cell.

In some embodiments, the frequency of the sinusoidal signal is provided at an energy sufficient to vibrate the malignant cell, the malignant cell having a transmissibility that is higher than a healthy cell.

In some embodiments, the listening environment is an indoor space. In some embodiments, each of the plurality of locations within the listening environment is a seating location within the listening environment.

In some embodiments, the method includes producing white Gaussian noise. In some embodiments, the method comprises modulating the one or more ultrasonic pressure waves by the Gaussian noise to produce one or more modulated noise signals. In some embodiments, the method includes transmitting, to the one or more target locations in the listening environment, the one or more modulated noise signals through the transmission medium.

In some embodiments, a method is provided for sound and therapeutic treatment in a listening environment which includes sampling sound by taking one or more sound samples from a listening environment; identifying a language, when present, inherent within audio information received from the one or more sound samples; producing an audio content signal from the audio information in the language; determining noise in the listening environment; producing a noise signal from the noise; producing an inverted noise signal by inverting the noise signal; generating a first modulated ultrasonic signal by modulating a first ultrasonic carrier with the inverted noise signal; generating a second modulated ultrasonic signal by modulating a second ultrasonic carrier with the audio content signal; and transmitting, to a target in the listening environment, an ultrasonic pressure wave, representative of the first modulated ultrasonic signal and the second modulated ultrasonic signal, through a transmission medium.

In some embodiments, the live translation can be controlled using a mobile application.

In some embodiments, each of the plurality of locations within the listening environment is a seating location within the listening environment.

In some embodiments, the audio content signal is produced, for an associated location from the audio information received from one or more sound samples, for each location within the listening environment, in a particular language.

In some embodiments, the frequency of the sinusoidal signal is provided at an energy sufficient to vibrate the malignant cell, the malignant cell having a transmissibility that is higher than a healthy cell.

In some embodiments, the live translation is controlled using a mobile application.

In some embodiments, a focused beam directional speaker system is provided that includes a noise detector; at least one microphone; a noise cancelling processor configured to produce a noise signal, representative of noise detected by the noise detector, and an inverse noise signal produced by inverting the noise signal; an audio processor configured to identify a language, when present, inherent within audio information received from the at least one microphone and to produce an audio content signal from audio information in the language; a summer configured to produce a combined input signal by summing the inverse noise signal and the audio content signal; a modulator configured to produce a modulated carrier signal by modulating an ultrasonic carrier signal with the combined input signal; at least one ultrasonic focused beam directional speaker configured to send, to a target in a listening environment, an ultrasonic pressure wave, representative of the modulated carrier signal, through a transmission medium, wherein in connection with the ultrasonic pressure wave reaching the target, the modulated carrier signal demodulates, thereby canceling noise and delivering the audio content signal to the target in the listening environment; and a sinusoidal signal generator, the sinusoidal signal generator being configured to provide one or more sinusoidal signals corresponding to a resonant frequency associated with a malignant cell.

In some embodiments, a master controller operatively controls, via a wireless link, the at least one ultrasonic focused beam directional speaker.

In some embodiments, the focused beam directional speaker system includes a master controller, operable to control the focused beam directional speaker system, coupled to the at least one ultrasonic focused beam directional speaker and the sinusoidal signal generator.

In some embodiments, the audio content is selected from the group consisting of noise-canceling sound, noise conditioning sound, and combinations thereof

In some embodiments, the frequency of the sinusoidal signal producing resonance in a malignant cell is selectable by a receiver of the therapeutic treatment.

### BRIEF DESCRIPTION OF THE DRAWINGS

Features, aspects, and advantages are described below with reference to the drawings, which are intended to illustrate, but not to limit, the invention. In the drawings, like reference characters denote corresponding features consistently throughout similar embodiments.

FIG. 1 illustrates a directed sound transmission system, which serves as an ultrasonic transducer that modulates audio information on an ultrasonic carrier.

FIG. 2 illustrates a schematic representation of directed sound transmission system 110 of FIG. 1.

FIG. 3 illustrates a perspective view of an outdoor audio system in use in an outdoor space.

FIG. 4 illustrates a block diagram of a sound system, including noise abatement.

FIG. 5 illustrates a perspective view of an indoor audio system operating in an indoor space.

FIG. 6 illustrates a perspective view of another example of an indoor audio system.

FIG. 7 illustrates a perspective view of a directed sound transmission system, including an indoor audio system.

FIG. 8 illustrates a perspective view of a directed sound source, including a sinusoidal sound source.

### DETAILED DESCRIPTION

Although certain embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be embodied as integrated or separate components.

For purposes of comparing various embodiments, certain aspects and advantages of these embodiments are described. All such aspects or advantages are not necessarily achieved by any particular embodiment. For example, various embodiments may be carried out in a manner that achieves or optimizes one advantage or group of advantages as taught herein without necessarily achieving other aspects or advantages as may also be taught or suggested herein.

Consumers want seamless integration of their smart devices within their homes, offices, and vehicles. With the existing technology, the only way to personalize audio content in a group setting is through headphone usage. The use of headphones and noise levels can have adverse effects on the health and well-being of users. The National Institutes of Health have found that five in ten young people listen to music too loudly, and 48 million people in the United States have trouble hearing (˜15% of the U.S. population).

Loudness is measured in a unit defined as decibels (dB). Noises that are above 85 dB may cause hearing loss over time by damaging ear fibers. The ear can repair itself if exposed to noise below a certain regeneration threshold, but once permanent damage occurs and one's hearing is gone, ear fibers cannot be fixed, nor can a person gain their hearing back. Some examples that employ a safe hearing range include whispering and normal conversations, which are around 30 dB and 60-80 dB, respectively. Unsafe zones include sporting events, rock concerts, and fireworks, which are about 94-110 dB, 95-115 dB, and 140-160 dB, respectively. Headphones fall into the range of 96-110 dB, placing them in the unsafe region. To give perspective, the ear should only be exposed to an intensity of 97 dB for about 3 hours per day, 105 dB for about 1 hour per day, or 110 dB for 30 minutes per day before causing ear damage.

As described, damage to the ear may occur when headphones deliver unsafe levels of sound directly to the ear canal. This damage is directly related to how much that sound makes your eardrum vibrate. When using speakers, sound waves have to travel a few feet before reaching the listener's ears. This distance allows some of the higher frequency waves to attenuate. With headphones, the eardrum will be excited by all frequencies without attenuation, so headphones can be more damaging than speakers at the same volume. Additionally, many people are trying to produce acoustic isolation when using headphones, which requires higher volumes to drown out ambient noise. For this reason, headphone audio levels should be chosen cautiously so as not to cause permanent ear damage and hearing loss.

In addition to hearing loss, headphones can cause a ringing in one or both ears, known as tinnitus, pain in the ear, or eardrum. Other physical effects from headphone use include ear infections, characterized by swelling, reddening, and discharge in the ear canal, itching pain, and feelings of tenderness or fullness in the ear. Impacted wax (i.e., wax buildup) and aural hygiene problems may also result from headphone use, as they can create a potential for bacteria to form in the ear canal due to increases in temperature and humidity of the ear canal.

The market is in dire need of a solution to ensure tranquility by delivering audio content with noise abatement without requiring the use of headphones. The current state of the technology is beneficial mainly for conversation enhancement and directional driver notifications from the vehicle but not focused on music or other audio input enhancement.

The present disclosure includes a parametric speaker system that revolutionizes how connected devices interact with in-vehicle audio systems. The parametric speaker system decentralizes sound to allow users to customize their in-vehicle audio content seat-by-seat. Ultimately, the system provides simple, connected entertainment for everyone.

After the user downloads and accesses a mobile application, the user may select a seat within an environment and take control of the speaker system for that seat. The mobile application collects the user and environmental data and sends it back to servers via a mobile connection.

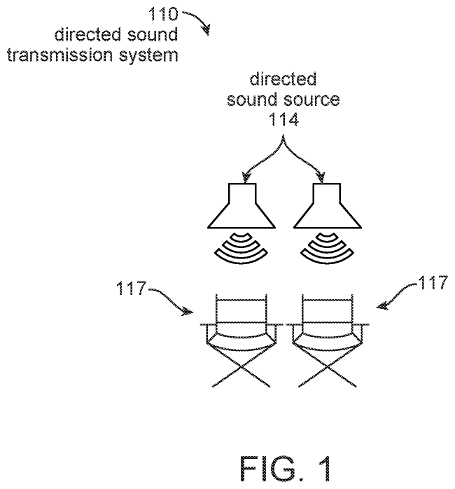

FIG. 1 illustrates directed sound transmission system 110, which serves as an ultrasonic transducer that modulates audio information on an ultrasonic carrier. In some examples, directed sound transmission system 110 may serve as an apparatus for the directed transmission of sound waves restricted to a particular listener within an environment. As illustrated in FIG. 1, directed sound transmission system 110 may be located within an environment, including directed sound source 114 and sinusoidal sound source 115, which may be situated in directed sound source 114. In some examples, at least one directed sound source 114 and one sinusoidal sound source 115 are installed in automobile 146 above and/or to the side of head 148 of listener 149, as shown in FIG. 1. Sinusoidal signal source 115 may direct a sinusoidal signal at one or more locations in a listening environment. The sinusoidal signal from the sinusoidal sound source need not necessarily be delivered on an ultrasonic carrier. The sinusoidal signal frequency may be applied with an intensity and driven at a frequency causing resonance of a given malignant cell. The malignant cell will vibrate and heat up, causing it to be torn apart. Frequencies within a range of frequencies may represent various resonant frequencies for various cancer cell types.

The application of known resonant frequencies may be selected by a user or programmed into a processor (not shown) for control sinusoidal sound source 115. Normal cells, being responsive to a different resonant frequency, will be largely unaffected. The sinusoidal signal may be attenuated by or reflected away from tissue and bones in the signal path between the signal source 115 and a possible point of application. Consequently, cancers on or near the skin's surface would be located in a position that would likely receive a more significant benefit from signal therapy than those located in a position more likely subject to attenuation or reflection. It should be noted that although FIG. 1 shows an application of directed sound waves via directed sound transmission system 110 over seat position(s) 117.

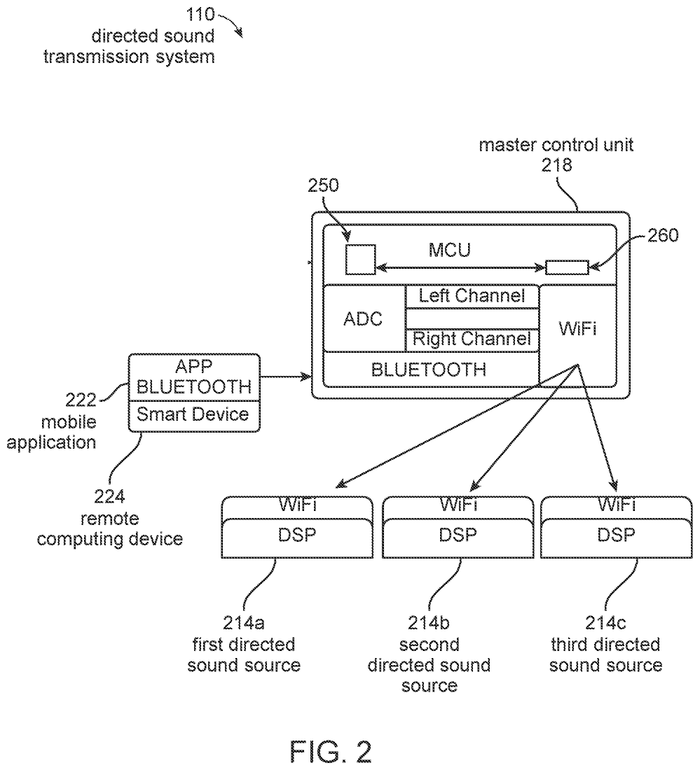

FIG. 2 illustrates a schematic representation of directed sound transmission system 110 of FIG. 1. In some examples, directed sound transmission system 110 (FIG. 1) includes an audio system 250, a master control unit (MCU) 218, a remote computing device 224 including a mobile application 222, and at least one directed sound source 114. FIG. 2 shows that, in some examples, the at least one directed sound source 114 (FIG. 1) includes a first directed sound source 214a, a second directed sound source 214b, and a third directed sound source 214c. Sinusoidal sound source 115 is also shown in FIG. 2.

In some examples, the MCU 218 comprises at least one processor 250 (e.g., an application processor) and at least one memory 260 having program instructions, that when executed by processor 250, are configured to cause directed sound transmission system 110 to direct sound as described herein. In some embodiments, memory 260 contains program instructions that, when executed by processor 250 (e.g., an open-source processor such as a 250 LinuxTM processor), cause directed sound transmission system 110 (FIG. 1) to direct sound as described herein.

In some examples, MCU 218 exists locally on a hardware system programmed according to program instruction downloaded from remote server 268. In other examples, MCU 218 exists on a hardware system located remotely with respect to elements of directed sound transmission system 110 according to program instruction downloaded from remote server 268.

In many examples, the multiple directed sound sources 214a, 214b, and 214c are communicatively coupled to the MCU 218. The MCU 218 allows for selecting a specific and different audio channel (e.g., right and left channel) for each of the at least one directed sound source 214 connected to the MCU 218, thus personalizing the content of each audio sound source. The target listener controls the content selection of this sound source by using their remote computing device 224. Accordingly, directed sound transmission system 110 may include more than one remote computing device 224. In many examples, directed sound transmission system 110 comprises one remote computing device 224 per listener. The remote computing device 224 may be configured to communicate with the MCU 218 via the mobile application 222 loaded on the remote computing device 224. In some examples, directed sound transmission system 110 (FIG. 1) contains a set of downloadable and installable software applications, mobile application 222, designed for retail smart devices, such as a remote computing device 224 (which may be, for example, a smartphone or tablet). Mobile application 222 runs on MCU 218, providing functionality to control at least one of (not shown) a seat selection, content selection, and source speaker volume (i.e., the volume of the applicable directed sound source) and sinusoidal source frequencies, per therapy selection, of sinusoidal source 115. Mobile application 222 may also identify a listener by a listener profile identification label (Profile ID). In some examples, usage data is collected and tagged with this Profile ID and stored in the cloud on remote server 268. The mobile application 222 may also provide firmware update functionality for the MCU 218 and the at least one directed sound source 214. Each directed sound source 214 (illustrated as 214a, 214b, and 214c) may include a digital signal processor (DSP) for controlling direct sound source operation. MCU 218 can also initiate and override the chosen content for each sound source connected to it.

In some examples, MCU 218 comprises a programmable computational module capable of executing software code and various interface modules for communication with external devices, such as a remote computing device 224. MCU 218 may include an analog-to-digital controller (ADC) for converting analog signals received in connection with implementing system operation. The interface modules may include WiFi, Bluetooth, and a control module configured to interface with an audio system (not explicitly shown).

Noise pollution is a significant concern for all environments. Installing directed sound transmission system 110 (FIG. 1) within environments may result in quieter spaces that would allow occupants to have their ideal experience and keep it isolated from the outside world.

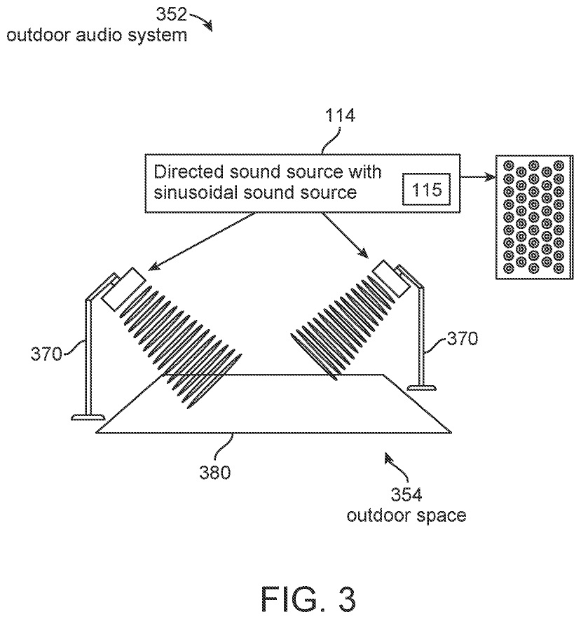

FIG. 3 illustrates a perspective view of outdoor audio system 352 in use in outdoor space 354. In some examples, outdoor audio system 352 may be representative of directed sound transmission system 110 (FIG. 1) and may provide transmission of sound waves within a confined location directed and restricted to a particular group of listeners. Stated differently, outdoor audio system 352 may be considered a noise abatement system. As illustrated in FIG. 3, outdoor audio system 352 includes at least one directed sound source 114 having sinusoidal sound source 115, which may be installed a few feet above the listener's head (not shown). Outdoor audio system 352, mounted on pole 370, as demonstrated in FIG. 3. Outdoor audio system 352 may also include a mechanism (not shown) to generate low-frequency sounds and vibrations (audio bass) located on floor 380 below a listeners' feet (not shown) in outdoor space 354.

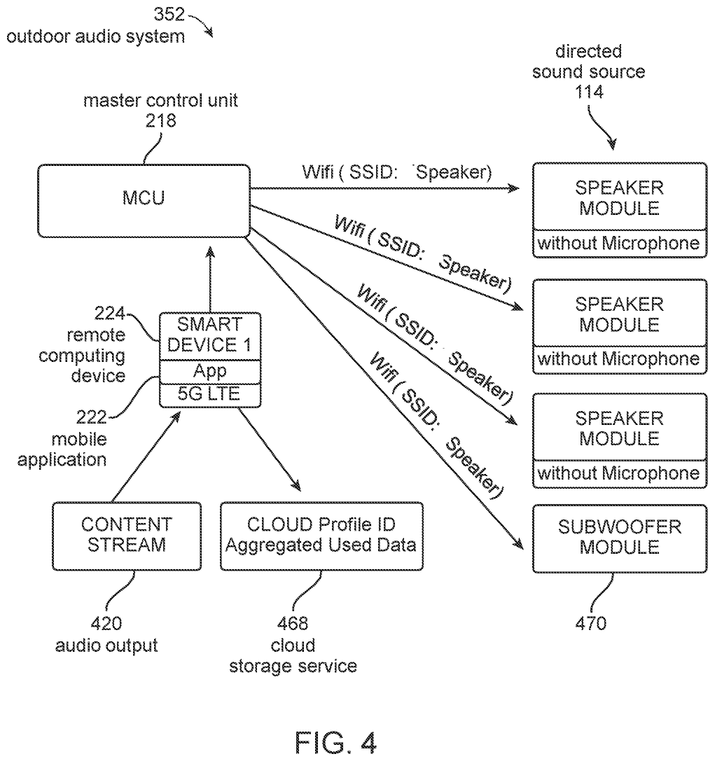

FIG. 4 illustrates a block diagram of a sound system, including noise abatement. As shown in FIG. 4, and as discussed with reference to directed sound transmission system 110, outdoor audio system 352 may include MCU 218 communicatively coupled (via, for instance, WiFi or a network identified in connection with a Service Set Identifier (S SID) to directed sound source 114, having a sinusoidal source 115, and a remote computing device 224 including mobile application 222. In some examples, outdoor audio system 352 contains a set of downloadable and installable software applications, mobile application 222, for use with retail smart devices, such as a remote computing device 224, which may be, for example, a smartphone or tablet. Mobile application 222, executed on MCU 218, provides functionality to control at least one of (not shown) a content selection and a source speaker volume (i.e., the volume of the applicable directed sound source). Mobile application 222 may also identify the listener by Profile ID. In some examples, usage data is collected and tagged via Profile ID and stored on the cloud in remote server 268. Mobile application 222 may also provide firmware update functionality for MCU 218 and directed sound source 114. In some examples, MCU 218 executes software for communication with external devices, such as a remote computing device 224 via a wireless network, such as WiFi or Bluetooth Outdoor audio system 352 may include a content stream (audio output 20) that is “fed” through remote computing device 224 to MCU 218. In addition to directed sound source 114, MCU 218 may be operatively coupled to subwoofer module 470. The sound abatement system and the examples described herein may use Gaussian white noise generated by a Gaussian noise generator (not shown) or implemented within MCU 218. Alternatively, the sound abatement system and examples described herein may use a noise cancellation system provided by a separate system or implemented within MCU 218. Sinusoidal source sound therapy may be delivered in connection with one or more speaker modules shown in FIG. 4.

Outdoor audio system 352 may provide directed sound transmission by modulating an ultrasonic carrier with sound. In connection with the modulated carrier striking a physical object such as the listener's head and ears, it demodulates, leaving audible sound for the listener to hear. The foregoing describes the delivery of sound herein in connection with directed sound transmission system 110 as described throughout. The therapeutic sound from sinusoidal source 115 may be delivered by a speaker (not shown), separate from the directed sound transmission system 110.

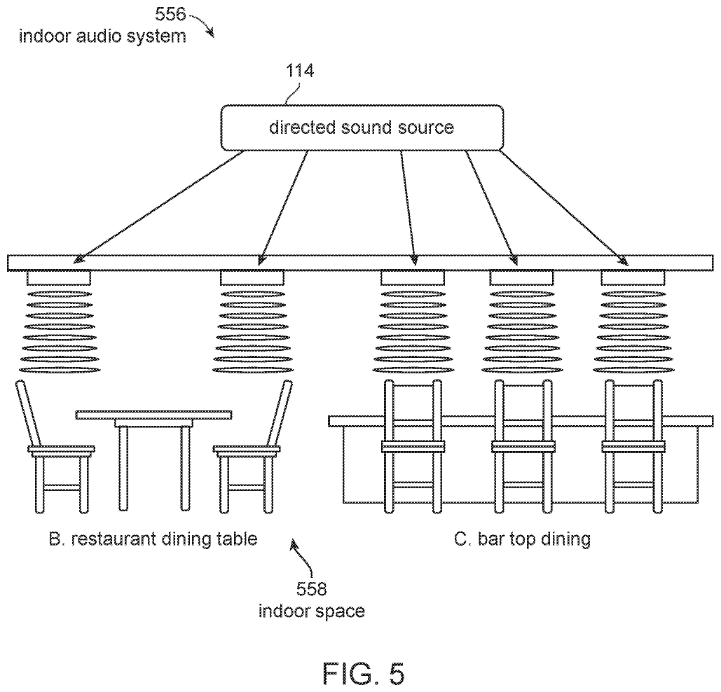

FIG. 5 illustrates a perspective view of indoor audio system 556, operating in indoor space 558, similar to outdoor audio system 352 of FIG. 3. In some examples, indoor space 558 includes a restaurant and/or bar setting. Indoor audio system 556 may direct sound waves to a particular listener or group of listeners within restaurants and bars. As shown in FIG. 5, indoor audio system 556 may include directed sound source 114 and sinusoidal sound source 115 installed above and/or to the side of the listeners' head.

While in indoor space 558, listeners may also select frequencies for the sinusoidal sound source that correspond to resonant frequencies destructive of associated malignant cells. Such therapy may provide a proactive measure against, for instance, skin cancers.

Providing directed sound source 114 in an environment may enable consumers to customize their music/audio at a booth and set the mood to which they desire.

Establishments like Sports Bars and Restaurants are known for entertaining patrons with multiple media options to enhance the overall experience. Many bars and restaurants have multiple T.V.s spread across the space, all showing different media. Typically, there is a single output audio source coming from a “master TV.” Indoor audio system 556 may provide seat-by-seat audio (via directed sound source 114) and source (tv) connectivity for each individual T.V. input. In some embodiments, a Quick Response (Q.R.) code will prompt a user to download mobile application 222 on their remote computing device 224 and show the user how the system functions, enabling the user to choose whichever input they prefer. Additional inputs may be integrated, such as jukebox libraries that can play seat by seat or table by table. Mobile application 222 allows collecting data on what users are streaming seat by seat through the connection to directed sound source 114. In addition, indoor audio system 556 may allow patrons to order food and drinks vocally and avoid touching high trafficked table-side ordering devices, thus enabling patrons to engage in the increasingly popular practice of contactless dining/ordering.

Indoor audio system 556 may also give retailers the unique opportunity to provide product placement voice information near (or near) their displays. Having employees push the same product information or specials to everyone who enters the store is repetitive and draining to the employee and disruptive to everyone's shopping experience. With the targeted placement of indoor audio system 556 in various locations around a store, customers can get individualized notifications, leaving sales associates with the bandwidth to support customer needs.

Exhibitions and conferences tend to have boisterous, exciting atmospheres. Every business, company, or entrepreneur is trying to grab and hold your attention. However, with multiple speaker systems going, it becomes hard to focus on any one exhibit. Introducing indoor audio system 556 to this environment will allow exhibits to set the mood for their individual presentation without distracting others.

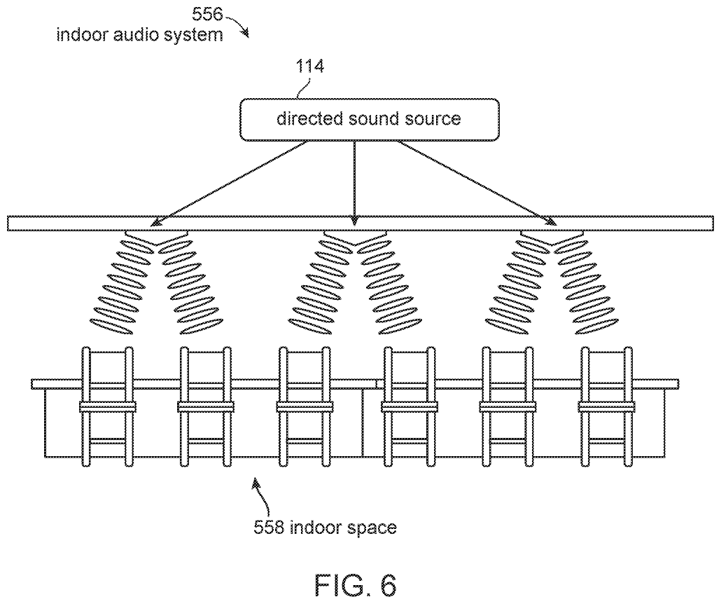

FIG. 6 illustrates a perspective view of another example of indoor audio system 556. FIG. 6 shows an installation point in the ceiling above a table, as shown in FIG. 6, or the ceiling above a more expansive area, including table seating, as shown in FIG. 5. Indoor audio system 556 may also include a mechanism to generate low-frequency sounds and vibrations (audio bass) located in the seat base and/or seat back at the listener's location. In some embodiments, indoor audio system 556 includes CU 218 (FIG. 4), coupled with remote computing device 224 (FIG. 4), allowing a user to control content selection. MCU 218 (FIG. 4) may also be communicatively coupled to directed sound source 114, including sinusoidal sound source 115. Allowing for control of content selection may include allowing each user to select a specific and different audio channel for each directed sound source 114, thereby personalizing the content of each directed sound source for a user or group of users.

In some embodiments, indoor audio system 556 contains a set of downloadable and installable software applications, the mobile application 222 (FIG. 4), designed for retail smart devices, such as remote computing device 224 (FIG. 4), which may be, for example, a smartphone or tablet. The mobile application 222 (FIG. 4) through MCU 218 (FIG. 4) provides functionality to control at least one of content selection and source speaker volume (i.e., the volume of the applicable directed sound source). The mobile application 222 (FIG. 4) may also be configured to identify a listener by Profile ID. In some examples, usage data is collected and tagged with a Profile ID and stored via cloud storage.

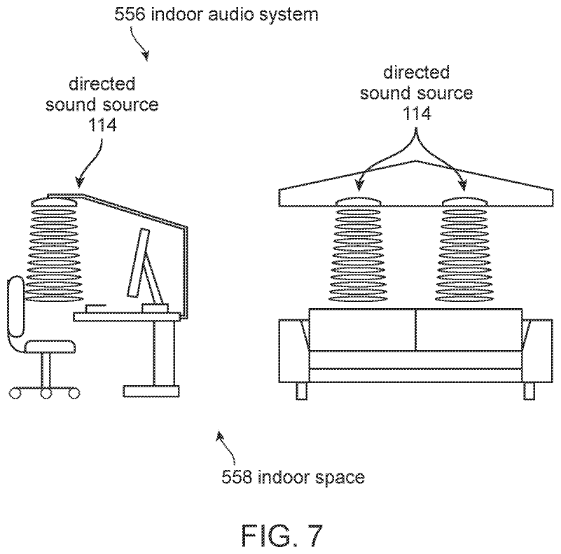

FIG. 7 illustrates a perspective view of another example of directed sound transmission system 110, including indoor audio system 556. Indoor audio system 556 may direct sound waves to a particular listener or group of listeners. As shown in FIG. 7, indoor audio system 556 may include directed sound source 114 (including sinusoidal sound source 115) that may be installed above and/or to the side of each listeners' head. One example of an installation location for indoor audio system 556 is a ceiling above a couch or over a desk, as shown in FIG. 7. Indoor audio system 556 may further include a mechanism to generate low-frequency sounds and vibrations (audio bass) located in the seat base and/or seat back at the listener's location. Indoor audio system 556 may be configured to communicatively couple to MCU 218 (FIG. 4), which may operatively couple to remote computing device 224 (FIG. 4).

In some examples, indoor audio system 556 may contain a set of downloadable and installable software applications, mobile application 222 (FIG. 4), and designed-for retail smart devices, such as a remote computing device 224 (FIG. 4). The mobile application 222 (FIG. 4) may provide the functionality to control content selection and source speaker volume from directed sound source 114. The mobile application 222 (FIG. 4) may also be configured to identify a listener by Profile ID. In some examples, usage data is collected and tagged with a Profile ID and stored in a cloud server (not shown).

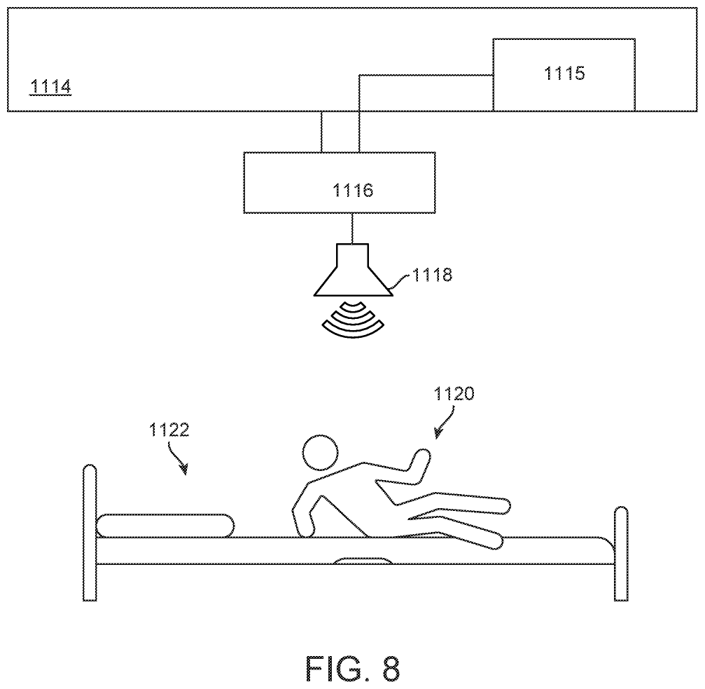

FIG. 8 illustrates a perspective view of directed sound source 1114, including sinusoidal sound source 1115. Speaker system 1116 may include a parametric speaker. User 1120 may receive, while resting on bed 1122, both noise-canceling and/or noise conditioning sound through a parametric speaker of speaker system 1116 in addition to the therapeutic sinusoidal sound of frequencies corresponding to a resonant frequency identified for a particular malignant cell type. Multiple frequencies corresponding to resonant frequencies of various malignant cell types may be selected by user 1120 in connection with receiving the therapeutic sinusoidal sound.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

The various features and processes described above may be used independently or combined in various ways. All possible combinations and subcombinations are intended to fall within the scope of this disclosure. In addition, certain methods, events, states, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The term “adjacent” is used to mean “next to or adjoining”. For example, the disclosure includes “the at least one directed sound source is located adjacent a head of the user.” In this context, “adjacent a head of the user” is used to mean that the at least one directed sound source is located next to a head of the user. The placement of the at least one directed sound source in a ceiling above the user's head, such as in a vehicle ceiling, would fall under the meaning of “adjacent” as used in this disclosure.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A method for providing sound and therapeutic treatment in a listening environment, comprising:
modulating one or more ultrasonic pressure waves by audio content to produce one or more modulated carrier signals;
sending the one or more modulated carrier signals, to one or more target locations in the listening environment, through a transmission medium, wherein in connection with the one or more ultrasonic pressure waves reaching the one or more target locations, the one or more modulated carrier signals demodulate; and
directing a sinusoidal signal at the one or more target locations in the listening environment, wherein a frequency of the sinusoidal signal produces resonance in a malignant cell.

2. The method of claim 1, wherein the frequency of the sinusoidal signal is provided at an energy sufficient to vibrate the malignant cell, the malignant cell having a transmissibility higher than a healthy cell.

3. The method of claim 1, wherein the listening environment is an indoor space.

4. The method of claim 3, wherein each of the plurality of locations within the listening environment is a seating location within the listening environment.

5. The method of claim 1, further comprising:
producing white Gaussian noise; and
modulating the one or more ultrasonic pressure waves by the Gaussian noise to produce one or more modulated noise signals.

6. The method of claim 5, further comprising transmitting, to the one or more target locations in the listening environment, the one or more modulated noise signals through the transmission medium.

7. The method of claim 1, further comprising:
sampling sound by taking one or more sound samples from a listening environment; and
identifying a language, when present, inherent within audio information received from the one or more sound samples.

8. The method of claim 7, further comprising:
producing an audio content signal from the audio information in the language; and
determining noise in the listening environment.

9. The method of claim 8, further comprising:
producing a noise signal from the noise;
producing an inverted noise signal by inverting the noise signal; and
generating a first modulated ultrasonic signal by modulating a first ultrasonic carrier with the inverted noise signal.

10. The method of claim 9, further comprising:
generating a second modulated ultrasonic signal by modulating a second ultrasonic carrier with the audio content signal; and
transmitting, to a target in the listening environment, an ultrasonic pressure wave, representative of the first modulated ultrasonic signal and the second modulated ultrasonic signal, through a transmission medium.

11. The method of claim 10, further comprising controlling the live translation using a mobile application.

12. The method of claim 10, wherein the target location is one of a plurality of seat positions in the listening environment.

13. The method of claim 10, wherein the audio content signal is produced, for an associated location from the audio information received from one or more sound samples, for each location within the listening environment, in the language.

14. The method of claim 13, wherein the frequency of the sinusoidal signal is provided at an energy sufficient to vibrate the malignant cell, the malignant cell having a transmissibility higher than a healthy cell.

15. The method of claim 14, further comprising controlling directional sound transmission using a mobile application.

16. A focused beam directional speaker system, comprising:
a noise detector;
at least one microphone;
a noise-canceling processor configured to produce a noise signal, representative of noise detected by the noise detector, and an inverse noise signal produced by inverting the noise signal;
an audio processor configured to identify a language, when present, inherent within audio information received from the at least one microphone and to produce an audio content signal from audio information in the language;
a summer configured to produce a combined input signal by summing the inverse noise signal and the audio content signal;
a modulator configured to produce a modulated carrier signal by modulating an ultrasonic carrier signal with the combined input signal;
at least one ultrasonic focused beam directional speaker configured to send, to a target in a listening environment, an ultrasonic pressure wave, representative of the modulated carrier signal, through a transmission medium, wherein in connection with the ultrasonic pressure wave reaching the target, the modulated carrier signal demodulates, thereby canceling noise and delivering the audio content signal to the target in the listening environment; and
a sinusoidal signal generator, the sinusoidal signal generator being configured to provide one or more sinusoidal signals corresponding to a resonant frequency associated with a malignant cell.

17. The focused beam directional speaker system of claim 16, wherein the master controller operatively controls, via a wireless link, the at least one ultrasonic focused beam directional speaker.

18. The focused beam directional speaker system of claim 16, wherein the master controller operatively controls, via a wired link, the at least one ultrasonic focused beam directional speaker and the sinusoidal signal generator.

19. The focused beam directional speaker system of claim 16, wherein the audio content is selected from the group consisting of noise-canceling sound, noise conditioning sound, and combinations thereof

20. The focused beam directional speaker system of claim 16, wherein the frequency of the sinusoidal signal producing resonance in a malignant cell is selectable by a receiver of the therapeutic treatment.
